(19) United States
(12) Patent Application Publication
(10) Pub. No.: US 2024/0327395 A1
(43) Pub. Date: Oct. 3, 2024

(54) A PROCESS FOR PREPARING BENZOXAZINONE COMPOUNDS

(71) Applicant: Tagros Chemicals India Pvt Ltd, Chennai, Tamil Nadu (IN)

(72) Inventors: Srikrishnan RAJAIAH, Chennai, Tamil Nadu (IN); Dinesh MURUGAN, Chennai, Tamil Nadu (IN); R. KUPPUSWAMY, Chennai, Tamil Nadu (IN)

(21) Appl. No.: 18/681,685

(22) PCT Filed: Aug. 11, 2022
(86) PCT No.: PCT/IN2022/050723

§ 371 Date: Feb. 6, 2024

(30) Foreign Application Priority Data
Nov. 2, 2021 (IN) 202141050378

(51) Int. Cl. C07D 413/14

(52) U.S. Cl. CPC C07D 413/14

(57) ABSTRACT

The present disclosure provides a process for preparing a compound of Formula I, the process comprising: a. mixing a compound of Formula II with a compound of Formula III in a mole ratio range of 1:1 to 1.3:1 to obtain a first mixture; b. adding a solvent to the first mixture to obtain a second mixture followed by cooling the second mixture; c. contacting a scavenger with the second mixture to obtain a reaction mass; and d. heating the reaction mass followed by addition of a reagent to obtain the compound of Formula I, wherein X is selected from Cl, Br, I, or CN; and the scavenger in step c and the reagent in step d are added in single lot.

### FIELD OF INVENTION

The present disclosure broadly relates to benzoxazinone compounds of Formula I. The present disclosure, in particular, relates to a process for preparing the compounds of Formula I.

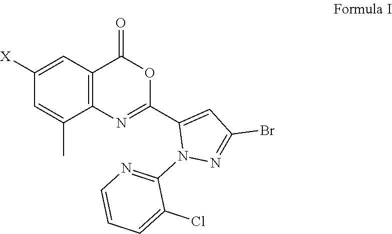

### BACKGROUND OF INVENTION

Benzoxazinone compounds are an interesting class of compounds that finds use either directly or indirectly in industrial, agricultural, and clinical applications. These heterocyclic compounds are used as starting materials for the preparation of various commercially valuable products in various fields. In particular, these compounds play a significant role in the synthesis of chlorantraniliprole, which is an effective insecticide for the control of a broad spectrum of pests.

WO03015519 provides a process for preparing a benzoxazinone compound which is an intermediate used in the preparation of insecticides. WO2004011453A2 provides a method for preparing 3-halo-4,5-dihydro-1H-pyrazole compounds which are useful intermediates in the preparation of the benzoxazinone compounds. WO2004111030 provides a method for preparing a fused oxazinone in which a carboxylic acid is contacted with a sulfonyl chloride and an isatoic anhydride in the presence of a tertiary amine. WO2004011447 provides a method for preparing a fused oxazinone in which a carboxylic acid is contacted with a sulfonyl chloride in the presence of an optionally substituted pyridine compound. CN101337959A provides an o-anisidine N-oxyl benzamide compounds which have insecticide effect and a preparation method for the same. WO2004033468 also discloses N-oxide compounds that are agronomic compounds, and their preparation methods.

However, the available preparation methods for benzoxazinone compounds involve tedious processes which in turn makes them a less approachable preparation process. Also, the processes developed incur long reaction time and are not easily scalable. Further, the reaction processes required the addition of reagents in multiple sequences which makes the preparation process difficult to handle and complicated. Hence in light of this, there is still a need in the state of art for a simple, one-pot preparation process of benzoxazinones which in turn are useful intermediates for the preparation of agrochemicals.

### SUMMARY OF THE INVENTION

In an aspect of the present disclosure, there is provided a process for preparing a compound of Formula I,

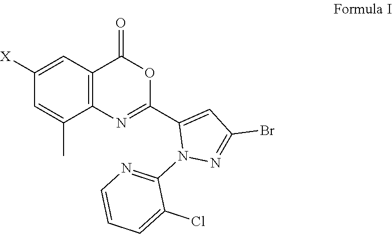

the process comprising: a) mixing a compound of Formula II with a compound of Formula III in a mole ratio range of 1:1 to 1.3:1 to obtain a first mixture; b) adding a solvent to the first mixture to obtain a second mixture followed by cooling the second mixture; c) contacting a scavenger with the second mixture to obtain a reaction mass; and d) heating the reaction mass followed by addition of a reagent to obtain the compound of Formula I, wherein X is selected from Cl, Br, I, or CN; and the scavenger in step c and the reagent in step d are added in a single lot.

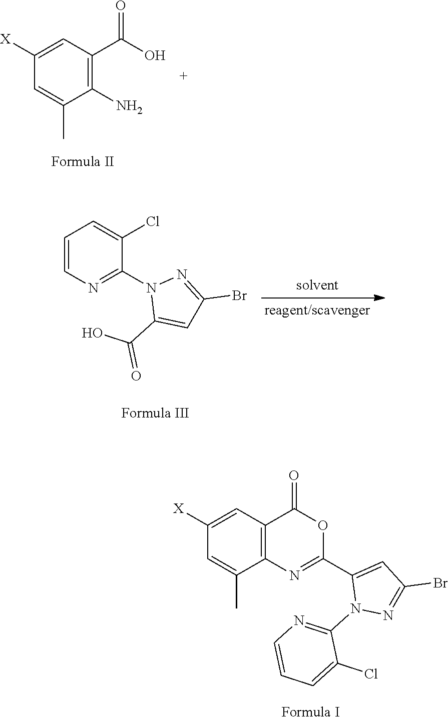

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “scavenger” used herein refers to a nitrogen containing compound, which is primarily a tertiary amine and is selected from triethyl amine, 3-methyl pyridine, 2-methyl pyridine, pyridine, and 4-dimethylaminopyridine. The process of the present disclosure produces acid as a byproduct and the reaction product needs to be protected from decomposition or other side reactions due to this acid. Hence, this acid byproduct needs to be scavenged. For this purpose, the amines are added to the reaction mixture which will generate an ammonium salt with the acid byproduct and is then removed. These scavengers are nitrogen containing compounds that can act as a base and as a scavenger as well.

The term “reagent” used herein refers to a substance that facilitates the reaction between the compounds of Formula II and Formula III to result in the compounds of Formula I. The reagent also facilitates the ring closure reaction in the formation of the compounds of Formula I. The reagent in the present disclosure includes but not limited to methane sulfonyl chloride, tosyl chloride, or oxalyl chloride.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a temperature range of 0 to 10° C. should be interpreted to include not only the explicitly recited limits of 0 to 10° C. but also to include sub-ranges, such as 0 to 9° C., 1 to 8° C., 0 to 5° C., and so forth, as well as individual amounts, including fractional amounts, within the specified ranges, such as 0° C., 1° C., 2° C., 3.5° C., 5° C., 7° C. and so on.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

As discussed in the background, the limitations in the processes for preparing benzoxazinones need to be addressed to arrive at a simple yet efficient preparation process. Also, there is a need to obtain compounds of high purity as these compounds are starting materials for the preparation of agrochemicals. It is to be noted that the purity of the intermediates largely affects the purity of the agrochemicals. Hence it is necessary to develop a process that is easy to manage as well provides higher yield and higher purity. Also, the process should be adaptable for large scale production. Accordingly, the present disclosure provides a process for preparing benzoxazinone compounds of Formula I which involves single step addition of the reacting materials and the reagents. The process of the present disclosure illustrates one pot synthetic procedure for the preparation of compounds of Formula I. The process involves one time addition of the reagent and the scavenger which makes the process less complicated and easy to handle. Further, the process of the present disclosure is carried out at an ambient temperature and pressure which makes the process more energy saving. Additionally, the completion of the reaction is ensured within the stipulated time period and requires less reaction time than the conventional processes. Accordingly, the present disclosure provides a process for preparing the compounds of Formula I comprising reacting compounds of Formula II with the compounds of Formula III in the presence of a reagent and a scavenger at ambient temperature. The process is also carried out using various reagents and scavengers, thereby providing a wide range of use in the preparation process. Thus, the process of the present disclosure is an economically viable process that can be extended to the industrial scale and at the same time maintain high yield and high purity.

The present disclosure is not to be limited in scope by the specific embodiments described herein, which are intended for the purposes of exemplification only. Functionally-equivalent products, compositions, and methods are clearly within the scope of the disclosure, as described herein.

In an embodiment of the present disclosure, there is provided a process for preparing a compound of Formula I,

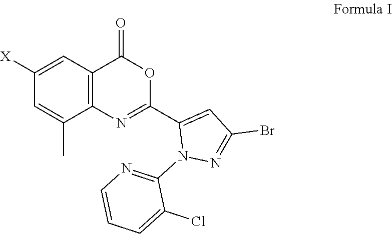

the process comprising:

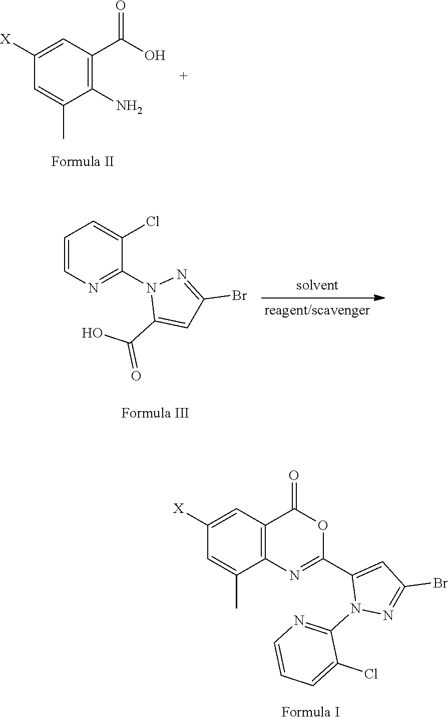

a) mixing a compound of Formula II with a compound of Formula III in a mole ratio range of 1:1 to 1.3:1 to obtain a first mixture; b) adding a solvent to the first mixture to obtain a second mixture followed by cooling the second mixture; c) contacting a scavenger with the second mixture to obtain a reaction mass; and d) heating the reaction mass followed by addition of a reagent to obtain the compound of Formula I, wherein X is selected from Cl, Br, I, or CN.

In an embodiment of the present disclosure, there is provided a process for preparing a compound of Formula I as disclosed herein, wherein mixing a compound of Formula II with a compound of Formula III is carried out in a mole ratio range of 1.1:1 to 1.3:1 to obtain a first mixture. In another embodiment of the present disclosure, wherein mixing a compound of Formula II with a compound of Formula III is carried out in the mole ratio of 1.2:1 to obtain a first mixture.

In an embodiment of the present disclosure, there is provided a process for preparing a compound of Formula I as disclosed herein, wherein the solvent is selected from acetonitrile, ethylene dichloride, tetrahydrofuran, ethyl acetate, acetone, dimethyl formamide, or combinations thereof. In another embodiment of the present disclosure, wherein the solvent is selected from acetonitrile, acetone, dimethyl formamide, or combinations thereof. In yet another embodiment of the present disclosure, wherein the solvent is acetonitrile.

In an embodiment of the present disclosure, there is provided a process for preparing a compound of Formula I as disclosed herein, wherein the scavenger is selected from triethyl amine, 3-methyl pyridine, 2-methyl pyridine, pyridine, 4-dimethylaminopyridine, or combinations thereof. In another embodiment of the present disclosure, wherein the scavenger is selected from 3-methyl pyridine, 2-methyl pyridine, pyridine, or 4-dimethylaminopyridine.

In an embodiment of the present disclosure, there is provided a process for preparing a compound of Formula I as disclosed herein, wherein the reagent is selected from methane sulfonyl chloride, tosyl chloride, or oxalyl chloride. In another embodiment of the present disclosure, wherein the reagent is oxalyl chloride. In one another embodiment of the present disclosure, wherein the reagent is methane sulfonyl chloride. In another embodiment of the present disclosure, wherein the reagent is tosyl chloride.

In an embodiment of the present disclosure, there is provided a process for preparing a compound of Formula I as disclosed herein, wherein cooling the second mixture is carried out at a temperature in a range of 0 to 10° C.

In another embodiment of the present disclosure, wherein cooling the second mixture is carried out at a temperature in a range of 0 to 7° C. In one another embodiment of the present disclosure, wherein cooling the second mixture is carried out at a temperature in a range of 0 to 5° C.

In an embodiment of the present disclosure, there is provided a process for preparing a compound of Formula I as disclosed herein, wherein heating the reaction mass is carried out at a temperature in a range of 10 to 20° C. In another embodiment of the present disclosure, wherein heating the reaction mass is carried out at a temperature in a range of 10 to 17° C. In one another embodiment of the present disclosure, wherein heating the reaction mass is carried out at a temperature in a range of 10 to 15° C.

In an embodiment of the present disclosure, there is provided a process for preparing a compound of Formula I as disclosed herein, wherein the reagent is slowly added to the reaction mass for a time period in a range of 1 to 2.5 hours. In another embodiment of the present disclosure, wherein the reagent is slowly added to the reaction mass for a time period in a range of 1.5 to 2.2 hours. In one another embodiment of the present disclosure, wherein the reagent is slowly added to the reaction mass for a time period of 2 hours.

In an embodiment of the present disclosure, there is provided a process for preparing a compound of Formula I as disclosed herein, wherein the compound of Formula III, the scavenger and the reagent are in a mole ratio range of 1:4:2 to 1:6:2.5. In another embodiment of the present disclosure, wherein the compound of Formula III, the scavenger and the reagent are in a mole ratio range of 1:4:2.2 to 1:6:2.5. In one another embodiment of the present disclosure, wherein the compound of Formula III, the scavenger and the reagent are in a mole ratio range of 1:4.8:2.3 to 1:5.1:2.5.

In an embodiment of the present disclosure, there is provided a process for preparing a compound of Formula I, the process comprising: a) mixing a compound of Formula II with a compound of Formula III in a mole ratio range of 1:1 to 1.3:1 to obtain a first mixture; b) adding a solvent selected from acetonitrile, ethylene dichloride, tetrahydrofuran, ethylacetate, acetone, dimethyl formamide, or combinations thereof, to the first mixture to obtain a second mixture followed by cooling the second mixture at a temperature in a range of 0 to 10° C.; c) contacting a scavenger selected from triethyl amine, 3-methyl pyridine, 2-methyl pyridine, pyridine, 4-dimethylaminopyridine, or combinations thereof with the second mixture to obtain a reaction mass; and d) heating the reaction mass at a temperature in a range of 10 to 20° C. followed by addition of a reagent selected from methane sulfonyl chloride, tosyl chloride, or oxalyl chloride to obtain the compound of Formula I, wherein X is selected from Cl, Br, I, or CN; the scavenger in step c and the reagent in step d are added in single lot and the compound of Formula III, the scavenger and the reagent are in a mole ratio range of 1:4:2 to 1:6:2.5.

In an embodiment of the present disclosure, there is provided a process for preparing a compound of Formula I, the process comprising: a) mixing a compound of Formula II with a compound of Formula III in a mole ratio range of 1:1 to 1.3:1 to obtain a first mixture; b) adding a solvent selected from acetonitrile, ethylene dichloride, tetrahydrofuran, ethylacetate, acetone, dimethyl formamide, or combinations thereof, to the first mixture to obtain a second mixture followed by cooling the second mixture at a temperature in a range of 0 to 10° C.; c) contacting a scavenger selected from triethyl amine, 3-methyl pyridine, 2-methyl pyridine, pyridine, 4-dimethylaminopyridine, or combinations thereof with the second mixture to obtain a reaction mass; and d) heating the reaction mass at a temperature in a range of 10 to 20° C. followed by addition of oxalyl chloride as a reagent to obtain the compound of Formula I, wherein X is selected from Cl, Br, I, or CN; the scavenger in step c and the reagent in step d are added in single lot and the compound of Formula III, the scavenger and the reagent are in a mole ratio range of 1:4:2 to 1:6:2.5.

In an embodiment of the present disclosure, there is provided a process for preparing a compound of Formula I, the process comprising: a) mixing a compound of Formula II with a compound of Formula III in a mole ratio range of 1:1 to 1.3:1 to obtain a first mixture; b) adding a solvent selected from acetonitrile, ethylene dichloride, tetrahydrofuran, ethylacetate, acetone, dimethyl formamide, or combinations thereof, to the first mixture to obtain a second mixture followed by cooling the second mixture at a temperature in a range of 0 to 10° C.; c) contacting a scavenger selected from triethyl amine, 3-methyl pyridine, 2-methyl pyridine, pyridine, 4-dimethylaminopyridine, or combinations thereof with the second mixture to obtain a reaction mass; and d) heating the reaction mass at a temperature in a range of 10 to 20° C. followed by addition of methane sulfonyl chloride as a reagent to obtain the compound of Formula I, wherein X is selected from Cl, Br, I, or CN; the scavenger in step c and the reagent in step d are added in single lot and the compound of Formula III, the scavenger and the reagent are in a mole ratio range of 1:4:2 to 1:6:2.5.

In an embodiment of the present disclosure, there is provided a process for preparing a compound of Formula I, the process comprising: a) mixing a compound of Formula II with a compound of Formula III in a mole ratio range of 1:1 to 1.3:1 to obtain a first mixture; b) adding a solvent selected from acetonitrile, ethylene dichloride, tetrahydrofuran, ethylacetate, acetone, dimethyl formamide, or combinations thereof, to the first mixture to obtain a second mixture followed by cooling the second mixture at a temperature in a range of 0 to 10° C.; c) contacting a scavenger selected from triethyl amine, 3-methyl pyridine, 2-methyl pyridine, pyridine, 4-dimethylaminopyridine, or combinations thereof, with the second mixture to obtain a reaction mass; and d) heating the reaction mass at a temperature in a range of 10 to 20° C. followed by addition of tosyl chloride as a reagent to obtain the compound of Formula I, wherein X is selected from Cl, Br, I, or CN; the scavenger in step c and the reagent in step d are added in single lot and the compound of Formula III, the scavenger and the reagent are in a mole ratio range of 1:4:2 to 1:6:2.5.

In an embodiment of the present disclosure, there is provided a process for preparing a compound of Formula I as disclosed herein, wherein the compound of Formula I is obtained with yield in a range of 80 to 98%; and purity in a range of 90 to 99%. In another embodiment of the present disclosure, wherein the compound of Formula I is obtained with yield in a range of 85 to 98%; and purity in a range of 97 to 99%.

Although the subject matter has been described in considerable detail with reference to certain examples and implementations thereof, other implementations are possible.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may apply.

The present disclosure illustrates the process of preparation of compounds of Formula I that are benzoxazinones compounds. The process of the present disclosure is a cost effective process that involves simple sequential one time addition of the reacting materials and does not involve a complicated process. The reacting materials of the present disclosure such as the compound of Formula II, Formula III, the scavenger, and the reagent are added in a single lot, thereby providing an easy to handle process. The present disclosure provides an industrially scalable process under more feasible operational conditions. Accordingly, the following examples exemplify the steps involved in the preparation of the compounds of Formula I. The examples below provide a detailed process for the preparation of compounds of benzoxazinones as well as with varying reagents and scavenger.

### Abbreviations

ACN Acetonitrile
DMAP 4-Dimethylaminopyridine
HPLC High Performance Liquid Chromatography

### Materials and Methods:

For the purpose of the present disclosure, the following materials were used.
a) acetonitrile—LR (laboratory grade)
b) triethylamine—LR
c) methane sulfonyl chloride—LR
d) 3-methylpyridine (3-picoline)—LR
e) 2-methylpyridine (2-picoline)—LR
f) pyridine—LR
g) 4-dimethylaminopyridine—LR
h) tosyl chloride—LR
i) oxalyl chloride—LR

The purity of the obtained compound of Formula I was determined using HPLC (Agilent C18, 4.6×250 mm, 5 um USUXA38257) analyser.

Benzoxazinone compounds of Formula I of the present disclosure, prepared by the process of the present disclosure is represented in the Scheme below.

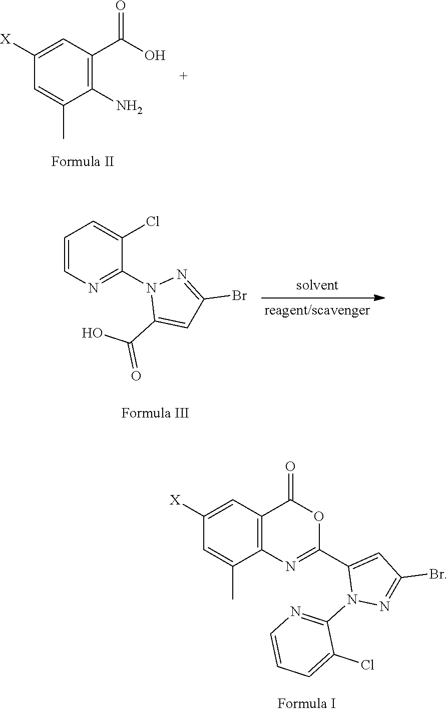

In Formula II, X is selected from Cl, Br, I, or CN. The solvent is selected from acetonitrile, ethylene dichloride, tetrahydrofuran, ethylacetate, acetone, or dimethyl formamide. The scavenger is selected from triethyl amine, 3-methyl pyridine, 2-methyl pyridine, pyridine, 4-dimethylaminopyridine, or combinations thereof. The reagent is selected from methane sulfonyl chloride, tosyl chloride, or oxalyl chloride.

### Compound 1: 2-(3-Bromo-1-(3-chloropyridin-2-yl)-1H-pyrazol-5-yl)-6-chloro-8-methyl-4H-benzo[d][1,3]oxazin-4-one

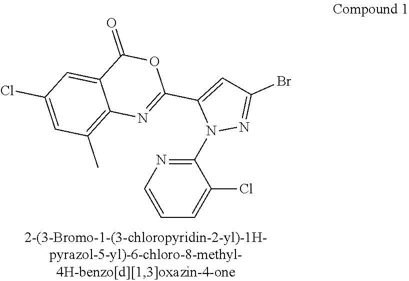 [SMILES: Cc1cc(Cl)cc2c(=O)oc(-c3cc(Br)nn3-c3ncccc3Cl)nc12]

### Compound 2: 6-bromo-2-(3-bromo-1-(3-chloropyridin-2-yl)-1H-pyrazol-5-yl)-8-methyl-4H-benzo[d][1,3]oxazin-4-one

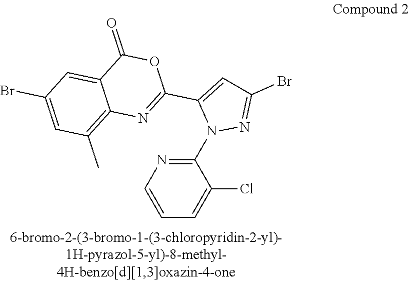 [SMILES: Cc1cc(Br)cc2c(=O)oc(-c3cc(Br)nn3-c3ncccc3Cl)nc12]

### Compound 3: 2-(3-bromo-1-(3-chloropyridin-2-yl)-1H-pyrazol-5-yl)-6-iodo-8-methyl-4H-benzo[d][1,3]oxazin-4-one

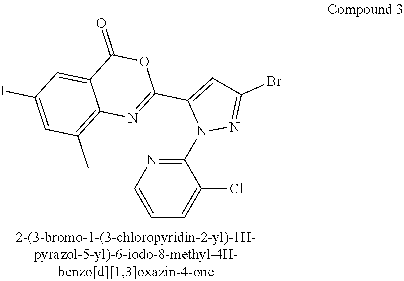 [SMILES: Cc1cc(I)cc2c(=O)oc(-c3cc(Br)nn3-c3ncccc3Cl)nc12]

### Compound 4: 2-(3-bromo-1-(3-chloropyridin-2-yl)-1H-pyrazol-5-yl)-8-methyl-4-oxo-4H-benzo[d][1,3]oxazine-6-carbonitrile

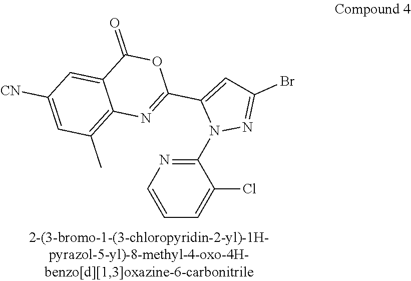

### Example 1

### Preparation of 2-(3-bromo-1-(3-chloropyridin-2-yl)-1H-pyrazol-5-yl)-6-chloro-8-methyl-4H-benzo[d][1,3]oxazin-4-one (Compound 1)

111.4 g (0.6 mole) of 2-amino-5-chloro-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile (solvent) was added to the first mixture to obtain a second mixture and then cooled to 0-5° C. 252.9 g (2.5 mole) of triethylamine (scavenger) was slowly added to the second mixture in the reactor over a period of 2 hrs, and was maintained for 30 min to obtain a reaction mass. The reaction mass was then slowly heated to 10-15° C. then 143.1 g (1.25 mole) of methane sulfonyl chloride (reagent) was added slowly to the reaction mass for a period of 2 hr and maintained for 2 hr at the same temperature. After the completion of reaction, the solvent acetonitrile was distilled out and then the crude mass of compound 1 obtained was quenched in 250 ml of water to get 199 g of solid compound 1 (crude yield 88%) with 95% area purity by HPLC after crystallization with ACN. The recovered solvent acetonitrile and triethylamine were recycled for the subsequent batches.

### Preparation of 6-bromo-2-(3-bromo-1-(3-chloropyridin-2-yl)-1H-pyrazol-5-yl)-8-methyl-4H-benzo[d][1,3]oxazin-4-one (Compound 2)

138.07 g (0.6 mole) of 2-amino-5-bromo-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture and cooled to 0-5° C. 232.8 g (2.5 mole) of 3-methylpyridine (scavenger) was slowly added to the second mixture in reactor over a period of 2 hr, maintained for 30 min to obtain a reaction mass. The reaction mass was slowly heated to 10-15° C. then 143.1 g (1.25 mole) of methane sulfonyl chloride (reagent) was added slowly to the reaction mass into the reactor for a period of 2 hr and maintained for 2 hr at the same temperature. After the completion of reaction, the solvent acetonitrile was distilled out then the crude mass of compound 2 obtained was quenched in 250 ml of water to get 238 g of solid compound 2 (crude yield 96%) with 98% area purity by HPLC after crystallization with ACN. The recovered solvent acetonitrile and 3-methylpyridine were recycled for the subsequent batches.

### Preparation of 2-(3-bromo-1-(3-chloropyridin-2-yl)-1H-pyrazol-5-yl)-6-iodo-8-methyl-4H-benzo[d][1,3]oxazin-4-one (Compound 3)

166.2 g (0.6 mole) of 2-amino-5-iodo-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture in the reactor to obtain a second mixture and cooled to 0-5° C. 232.8 g (2.5 mole) of 3-methylpyridine(scavenger) was slowly added to the second mixture in the reactor over a period of 2 hrs and was maintained for 30 min to obtain a reaction mass. The reaction mass was then slowly heated to 10-15° C. then 143.1 g (1.25 mole) of methane sulfonyl chloride (reagent) was slowly added to the reaction mass into the reactor for a period of 2 hr and maintained for 2 hr at the same temperature. After the completion of reaction, the solvent acetonitrile was distilled out and then the crude mass of compound 3 obtained was quenched in 250 ml of water to get 264 g of solid compound 3 (crude yield 97%) with 98% area purity by HPLC after crystallization with ACN. The recovered solvent acetonitrile and 3-methylpyridine were recycled for the subsequent batches

### Preparation of 2-(3-bromo-1-(3-chloropyridin-2-yl)-1H-pyrazol-5-yl)-8-methyl-4-oxo-4H-benzo[d][1,3]oxazine-6-carbonitrile (Compound 4)

105.73 g (0.6 mole) of 2-amino-5-cyano-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture to obtain a second mixture and cooled to 0-5° C. 232.8 g (2.5 mole) of 3-methylpyridine (scavenger) was slowly added to the second mixture into reactor over a period of 2 hrs, and was maintained for 30 min to obtain a reaction mass. The reaction mass was then slowly heated to 10-15° C. then 143.1 g (1.25 mole) of methane sulfonyl chloride (reagent) was slowly added to the reaction mass into the reactor for a period of 2 hr and maintained for 2 hr at the same temperature. After the completion of reaction, the solvent acetonitrile was distilled out and then the crude mass of compound 4 obtained was quenched in 250 ml of water to get 210 g of solid compound 4 (crude yield 95%) with 97% area purity by HPLC. The recovered solvent acetonitrile and 3-methylpyridine were recycled in the subsequent batches.

### Example 2

### Preparation of Compound 1 by varying the scavenger and/or the reagent

111.4 g (0.6 mole) of 2-amino-5-chloro-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture in the reactor to obtain a second mixture and cooled to 0-5° C. 232.8 g (2.5 mole) of 3-methylpyridine (scavenger) was slowly added to the second mixture in the reactor over a period of 2 hrs, and maintained for 30 min to obtain a reaction mass. The reaction mass was slowly heated to 10-15° C. then 143.1 g (1.25 mole) of methane sulfonyl chloride (reagent) was slowly added into the reactor for a period of 2 hrs and maintained for 2 hrs at the same temperature. After the completion of reaction, the solvent acetonitrile was distilled out and then the crude mass of compound 1 was quenched in 250 ml of water to get 220 g of solid compound 1 (crude yield 98%) with 95-97% area purity of HPLC after crystallization with ACN. The recovered solvent acetonitrile and 3-methylpyridine were recycled for the subsequent batches.

111.4 g (0.6 mole) of 2-amino-5-chloro-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture of the reactor to obtain a second mixture and cooled to 0-5° C. 232.8 g (2.5 mole) of 2-methylpyridine (scavenger) was slowly added to the second mixture in the reactor over a period of 2 hrs, and was maintained for 30 min to obtain a reaction mass. The reaction mass was slowly heated to 10-15° C. then 143.1 g (1.25 mole) of methane sulfonyl chloride (reagent) was slowly added into the reactor for a period of 2 hrs and maintained for 2 hrs at the same temperature. After the completion of reaction, the solvent acetonitrile was distilled out and then the crude mass of compound 1 obtained was quenched in 250 ml of water to get 216 g of solid compound 1 (crude yield 95.5%) with 98% area purity by HPLC after crystallization with ACN. The recovered solvent acetonitrile and 2-methylpyridine were recycled for the subsequent batches.

111.4 g (0.6 mole) of 2-amino-5-chloro-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture of the reactor to obtain a second mixture and cooled to 0-5° C. 197.7 g (2.5 mole) of pyridine (scavenger) was slowly added to the second mixture into reactor over a period of 2 hr and was maintained for 30 min to obtain a reaction mass. The reaction mass was slowly heated to 10-15° C. and then 143.1 g (1.25 mole) of methane sulfonyl chloride (reagent) was slowly added to the reaction mass into the reactor for a period of 2 hrs and maintained for 2 hrs at the same temperature. After the completion of reaction, the solvent acetonitrile was distilled out and then the crude mass of compound 1 was quenched in 250 ml of water to get 215 g of solid (crude yield 95%) with 97% area purity by HPLC after crystallization with ACN. The recovered solvent acetonitrile and pyridine were recycled for the subsequent batches.

111.4 g (0.6 mole) of 2-amino-5-chloro-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture in the reactor to obtain a second mixture and cooled to 0-5° C. 305.4 g (2.5 mole) of 4-dimethylaminopyridine (DMAP) (scavenger) was slowly added into reactor over a period of 2 hrs, and was maintained for 30 min to obtain a reaction mass. The reaction mass was slowly heated to 10-15° C. then 143.1 g (1.25 mole) of methane sulfonyl chloride (reagent) was added slowly into the reactor for a period of 2 hrs and maintained for 2 hrs at the same temperature. After the completion of reaction, the solvent acetonitrile was distilled out then the crude mass of compound 1 obtained was quenched in 250 ml of water to get 183 g of solid compound 1 (crude yield 81%) with 93% area purity by HPLC after crystallization with ACN. The recovered solvent acetonitrile and 4-dimethylaminopyridine (DMAP) were recycled for the subsequent batches.

111.4 g (0.6 mole) of 2-amino-5-chloro-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture in the reactor to obtain a second mixture and cooled to 0-5° C. 252.9 g (2.5 mole) of triethylamine (scavenger) was slowly added to the second mixture into the reactor over a period of 2 hrs and was maintained for 30 min to obtain a reaction mass. The reaction mass was slowly heated to 10-15° C. and then 238.16 g (1.25 mole) of tosyl chloride (reagent) was slowly added into the reactor for a period of 2 hrs and maintained for 2 hrs at the same temperature. After the completion of reaction, the solvent acetonitrile was distilled out and then the crude mass of compound 1 obtained was quenched in 250 ml of water to get 193 g of solid compound 1 (crude yield 85%) with 92% area purity by HPLC after crystallization with ACN. The recovered solvent acetonitrile and triethylamine were recycled for the subsequent batches.

111.4 g (0.6 mole) of 2-amino-5-chloro-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture of the reactor to obtain a second mixture and cooled to 0-5° C. 232.8 g (2.5 mole) of 3-picoline (3-methyl pyridine, scavenger) was slowly added to the second mixture into reactor over a period of 2 hrs and was maintained for 30 min to obtain a reaction mass. The reaction mass was slowly heated to 10-15° C. and then 238.16 g (1.25 mole) of tosyl chloride (reagent) was slowly added to the reaction mass into the reactor for a period of 2 hrs and maintained for 2 hrs at the same temperature. After the completion of reaction, the solvent acetonitrile was distilled out and then the crude mass of compound 1 obtained was quenched in 250 ml of water to get 208 g of solid compound 1 (crude yield 92%) with 96% area purity by HPLC after crystallization with ACN. The recovered solvent acetonitrile and 3-methylpyridine were recycled in the subsequent batches.

111.4 g (0.6 mole) of 2-amino-5-chloro-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture in the reactor to obtain a second mixture and cooled to 0-5° C. 232.8 g (2.5 mole) of 2-picoline(2-methyl pyridine, scavenger) was slowly added to the second mixture into reactor over a period of 2 hrs, and was maintained for 30 min to obtain a reaction mass. The reaction mass was slowly heated to 10-15° C. and then 238.16 g (2.5 eq, 1.25 mole) of tosyl chloride (reagent) was slowly added into the reactor for a period of 2 hrs and maintained for 2 hra at the same temperature. After the completion of reaction, the solvent acetonitrile was distilled out and then the crude mass of compound 1 obtained was quenched in 250 ml of water to get 203.5 g of solid compound 1 (crude yield 90%) with 94% area purity by HPLC after crystallization with ACN. The recovered solvent acetonitrile and 2-pyridine were recycled for the subsequent batches.

111.4 g (0.6 mole) of 2-amino-5-chloro-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture in the reactor to obtain a second mixture and cooled to 0-5° C. 197.7 g of pyridine (scavenger) was slowly added to the second mixture into reactor over a period of 2 hrs, and was maintained for 30 min to obtain a reaction mass. The reaction mass was slowly heated to 10-15° C. and then 238.16 g (1.25 mole) of tosyl chloride (reagent) was added slowly to the reaction mass into the reactor for a period of 2 hrs and maintained for 2 hrs at the same temperature. After the completion of reaction, the solvent acetonitrile was distilled out then the crude mass of compound 1 was quenched in 250 ml of water to get 206 g of solid (crude yield 91%) with 95% area purity by HPLC after crystallization with ACN. The recovered solvent acetonitrile and pyridine were recycled for the subsequent batches.

111.4 g (0.6 mole) of 2-amino-5-chloro-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture to obtain a second mixture and then cooled to 0-5° C. 305.4 g (2.5 mole) of 4-dimethylaminopyridine (DMAP) (scavenger) was slowly added to the second mixture in the reactor over a period of 2 hrs, and was maintained for 30 min to obtain a reaction mass. The reaction mass was slowly heated to 10-15° C. and then 238.16 g (1.25 mole) of tosyl chloride (reagent) was slowly added to the reaction mass for a period of 2 hrs and maintained for 2 hrs at the same temperature. After the completion of reaction, distilled out the solvent acetonitrile then the crude mass of compound 1 obtained was quenched in 250 ml of water to get 181 g of solid compound 1 (crude yield 80%) with 91% area purity by HPLC after crystallization with ACN. The recovered solvent acetonitrile and 4-dimethylaminopyridine (DMAP) were recycled for the subsequent batches.

111.4 g (0.6 mole) of 2-amino-5-chloro-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture to obtain a second mixture and cooled to 0-5° C. 197.7 g (2.5 mole) of pyridine (scavenger) was slowly added to the second mixture in the reactor over a period of 2 hrs, and was maintained for 30 min to obtain a reaction mass. The reaction mass was then slowly heated to 10-15° C. then 158.56 g (1.25 mole) of oxalyl chloride (reagent) was added slowly to the reaction mass into the reactor for a period of 2 hrs and maintained for 2 hrs at the same temperature. After the completion of reaction, the solvent acetonitrile was distilled out and then the crude mass of compound 1 obtained was quenched in 250 ml of water to get 206 g of solid (crude yield 91%) with 95% area purity by HPLC after crystallization with ACN. The recovered solvent acetonitrile and pyridine were recycled for the subsequent batches.

111.4 g (0.6 mole) of 2-amino-5-chloro-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture to obtain a second mixture and cooled to 0-5° C. 232.8 g (2.5 mole) of 3-picoline (3-methyl pyridine, scavenger) was slowly added to the second mixture in the reactor over a period of 2 hrs, and was maintained for 30 min to obtain a reaction mass. The reaction mass was then slowly heated to 10-15° C. then 158.56 g (1.25 mole) of oxalyl chloride (reagent) was added slowly to the reaction mass into the reactor for a period of 2 hrs and maintained for 2 hrs at the same temperature. After the completion of reaction, the solvent acetonitrile was distilled out and then the crude mass of compound 1 obtained was quenched in 250 ml of water to get 203 g of solid (crude yield 90%) with 94% area purity by HPLC after crystallization with ACN. The recovered solvent acetonitrile and picoline were recycled for the subsequent batches.

111.4 g (0.6 mole) of 2-amino-5-chloro-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture to obtain a second mixture and cooled to 0-5° C. 252.9 g (2.5 mole) of triethylamine (scavenger) was slowly added to the second mixture in the reactor over a period of 2 hrs, and was maintained for 30 min to obtain a reaction mass. The reaction mass was then slowly heated to 10-15° C. then 158.56 g (1.25 mole) of oxalyl chloride (reagent) was added slowly to the reaction mass into the reactor for a period of 2 hrs and maintained for 2 hrs at the same temperature. After the completion of reaction, the solvent acetonitrile was distilled out and then the crude mass of compound 1 obtained was quenched in 250 ml of water to obtain 194 g of solid (crude yield 86%) with 91% area purity of HPLC after crystallization with ACN. The recovered solvent acetonitrile and triethylamine were recycled for the subsequent batches.

### Example 3

### Preparation of Compound 2 with Oxalyl Chloride as Reagent and by Varying the Scavenger

138 g (0.6 mole) of 2-amino-5-bromo-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture to obtain a second mixture and cooled to 0-5° C. 197.7 g (2.5 mole) of pyridine (scavenger) was slowly added to the second mixture in the reactor over a period of 2 hrs, and was maintained for 30 min to obtain a reaction mass. The reaction mass was then slowly heated to 10-15° C. then 158.56 g (1.25 mole) of oxalyl chloride (reagent) was added slowly to the reaction mass into the reactor for a period of 2 hrs and maintained for 2 hrs at the same temperature. After the completion of reaction, the solvent acetonitrile was distilled out and then the crude mass of compound 2 obtained was quenched in 250 ml of water to get 218 g of solid (crude yield 88%) with 93% area purity in HPLC after crystallization with ACN. The recovered solvent acetonitrile and pyridine were recycled for the subsequent batches.

138 g (0.6 mole) of 2-amino-5-bromo-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture to obtain a second mixture and cooled to 0-5° C. 232.8 g (2.5 mole) of 3-picoline (3-methyl pyridine, scavenger) was slowly added to the second mixture in the reactor over a period of 2 hrs, and was maintained for 30 min to obtain a reaction mass. The reaction mass was then slowly heated to 10-15° C. then 158.56 g (1.25 mole) of oxalyl chloride was added slowly to the reaction mass into the reactor for a period of 2 hrs and maintained for 2 hrs at the same temperature. After the completion of reaction, the solvent acetonitrile was distilled out and then the crude mass of compound 2 obtained was quenched in 250 ml of water to get 229 g of solid (crude yield 89%) with 94% area purity by HPLC after crystallization with ACN. The recovered solvent acetonitrile and pyridine were recycled for the subsequent batches.

### Example 4

### Preparation of Compound 3 with Oxalyl Chloride as Reagent and by Varying the Scavenger

166.2 g (0.6 mole) of 2-amino-5-iodo-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture to obtain a second mixture and cooled to 0-5° C. 197.7 g (2.5 mole) of pyridine was slowly added to the second mixture in the reactor over a period of 2 hrs, and was maintained for 30 min to obtain a reaction mass. The reaction mass was then slowly heated to 10-15° C. then 158.56 g (1.25 mole) of oxalyl chloride (reagent) was added slowly to the reaction mass into the reactor for a period of 2 hrs and maintained for 2 hrs at the same temperature. After the completion of reaction, the solvent acetonitrile was distilled out and then the crude mass of compound 3 obtained was quenched in 250 ml of water to get 236 g of solid (crude yield 87%) with 92% area purity by HPLC after crystallization with ACN. The recovered solvent acetonitrile and pyridine were recycled for the subsequent batches.

166.2 g (0.6 mole) of 2-amino-5-iodo-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture to obtain a second mixture and cooled to 0-5° C. 232.8 g (2.5 mole) of 3-picoline (3-methyl pyridine, scavenger) was slowly added to the second mixture in the reactor over a period of 2 hrs, and was maintained for 30 min to obtain a reaction mass. The reaction mass was then slowly heated to 10-15° C. then 158.56 g (1.25 mole) of oxalyl chloride (reagent) was added slowly to the reaction mass into the reactor for a period of 2 hrs and maintained for 2 hrs at the same temperature. After the completion of reaction, the solvent acetonitrile was distilled out and then the crude mass of compound 3 obtained was quenched in 250 ml of water to get 239 g of solid (crude yield 89%) with 92% area purity by HPLC after crystallization with ACN. The recovered solvent acetonitrile and picoline were recycled for the subsequent batches.

### Example 5

### Preparation of Compound 4 with Oxalyl Chloride as Reagent and by Varying the Scavenger

105.7 g (0.6 mole) of 2-amino-5-cyano-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture to obtain a second mixture and cooled to 0-5° C. 197.7 g (2.5 mole) of pyridine (scavenger) was slowly added to the second mixture in the reactor over a period of 2 hrs, and was maintained for 30 min to obtain a reaction mass. The reaction mass was then slowly heated to 10-15° C. then 158.56 g (1.25 mole) of oxalyl chloride (reagent) was added slowly to the reaction mass into the reactor for a period of 2 hrs and maintained for 2 hrs at the same temperature. After the completion of reaction, the solvent acetonitrile was distilled out and then the crude mass of compound 4 obtained was quenched in 250 ml of water to get 176.5 g of solid (crude yield 80%) with 90% area purity by HPLC after crystallization with ACN. The recovered solvent acetonitrile and pyridine were recycled for the subsequent batches.

105.7 g (0.6 mole) of 2-amino-5-cyano-3-methylbenzoic acid (Formula II) and 151.3 g of (0.5 mole) 3-bromo-1-(3-chloropyridin-2-yl)-1H-pyrazole-5-carboxylic acid (Formula III) were mixed to obtain a first mixture and was taken in a reactor. 500 ml (472 g, 11.8 mole) of acetonitrile was added to the first mixture to obtain a second mixture and cooled to 0-5° C. 232.8 g (2.5 mole) of 3-picoline (3-methyl pyridine, scavenger) was slowly added to the second mixture in the reactor over a period of 2 hrs, and was maintained for 30 min to obtain a reaction mass. The reaction mass was then slowly heated to 10-15° C. then 158.56 g (1.25 mole) of oxalyl chloride (reagent) was added slowly to the reaction mass into the reactor for a period of 2 hrs and maintained for 2 hrs at the same temperature. After the completion of reaction, the solvent acetonitrile was distilled out and then the crude mass of compound 4 obtained was quenched in 250 ml of water to get 183 g of solid (crude yield 83%) with 92% area purity by HPLC after crystallization with ACN. The recovered solvent acetonitrile and picoline were recycled for the subsequent batches.

### Example 6

### Preparation of Compound-1 by Varying the Molar Ratio of Formula III and Formula II

The benzoxazinone of compound-1 was prepared according to the process provided in Example-1 by varying the molar ratio of 3-Bromo-1-(3-chloropyridin-2-yl)-1H-pyrazole-5-carboxylic acid and 2-amino-5-chloro-3-methylbenzoic acid as shown in Table-1 below.

TABLE 1

| | 3-Bromo-1-(3- | | | | |
|---|---|---|---|---|---|
| | chloropyridin-2-yl)-1H- | 2-Amino-5-chloro- | Methane | | |
| | pyrazole-5-carboxylic | 3-methylbenzoic | sulfonyl | | |
| | acid (mole ratio) | acid (mole ratio) | chloride | Yield | Purity |
| S. No | (Formula III) | (Formula II) | (mole ratio) | % | % |
| 1 | 1 | 0.9 | 2.5 | 62 | 95 |
| 2 | 1 | 1.1 | 2.5 | 92 | 96 |
| 3 | 1 | 1.2 | 2.5 | 98 | 96 |
| 4 | 1 | 1.3 | 2.5 | 86.2 | 94 |
| 5 | 1 | 1.4 | 2.5 | 68.5 | 93 |

From Table-1 the significance of the molar ratio of Formula II and Formula III in the range of 1:1 to 1.3:1 can be understood. When the mole ratio was less than 1:1, the purity was 95%, the yield drastically decreased to 62% Similarly when the ratio of the Formula II to Formula III was outside said range i.e., 1.4:1, the detrimental effect was observed towards the yield, i.e., yield decreased to 68.5%. On the other hand, when the ratio of the Formula II to Formula III is in said range of 1:1 to 1.3:1, it could be observed that both yield and the purity was higher. This confirmed that the process of the present disclosure was found to be efficient within the disclosed process parameters.

### Preparation of Compound 1 by Varying the Molar Ratio of Scavenger and Reagent

The benzoxazinone of compound-1 was prepared according to the method provided in the Example-1 above and by varying the molar ratio of scavenger (3-picoline) and the reagent (methane sulfonyl chloride) with respect to the mole ratio of compound of Formula III as shown in Table-2.

TABLE 2

| | 3-Bromo-1-(3- | | | | | |
|---|---|---|---|---|---|---|
| | chloropyridin-2- | | | | | |
| | yl)-1H-pyrazole- | 2-Amino-5- | Methane | | | |
| | 5-carboxylic acid | chloro-3- | sulfonyl | 3-Picoline | | |
| | (mole ratio) | methylbenzoic | chloride (mole | (mole ratio) | Yield | Purity |
| S. No | (Formula III) | acid (mole ratio) | ratio)(reagent) | (scavenger) | % | % |
| 1 | 1 | 1.2 | 2.4 | 5 | 85 | 90 |
| 2 | 1 | 1.2 | 2.5 | 5 | 98 | 96 |
| 3 | 1 | 1.2 | 2.6 | 5 | 70 | 93.7 |
| 4 | 1 | 1.2 | 2.5 | 4 | 89 | 92 |
| 5 | 1 | 1.2 | 2.5 | 6 | 98 | 95 |

The mole ratio of the compound of the Formula III to the scavenger and the reagent was maintained in said ratio range of 1:4:2 to 1:6:2.5. The processes carried out outside said ranges resulted in lower yield of the compound of Formula I. From Table 2 it could be observed, that when the reagent was employed in higher mole ratio, i.e., outside the disclosed range, then the yield of the compound of Formula I was found to be reduced to 70%. Therefore in order to obtain higher yield as well as higher purity of the compound of Formula I, the mole ratio of the reacting compounds of Formulae II, III, scavenger and reactants should be maintained well within the disclosed ratio range.

### Preparation of Compound 1 by Varying the Reaction Temperature

Further to understand the critical aspect regarding the process parameters, the process of preparation of the compound 1 of the present disclosure was carried out by varying the reaction temperature as shown in Table-3. The preparation follows the process as provided in Example-1 and by varying the temperature at which the reaction mass was heated. The same temperature was maintained while the reagent was added slowly and thus the reaction temperature was varied in the range of −5 to 35° C. From the Table-3 it could be identified that the process was found to be effective when the reaction temperature was in the range of 10 to 20° C. specifically when the temperature was in a range of 10 to 15° C. When the temperature was lowered or increased, the yield as well as the purity were adversely affected. The purity and yield of the compound of Formula I was maximum at a reaction cooking temperature of 10 to 15° C. Thus it is evident that the reaction temperature is also an essential feature to obtain the compound of Formula-I at a higher yield and higher purity.

TABLE 3

| | 3-Picoline | | Reaction | | |
|---|---|---|---|---|---|
| | addition | Methane sulfonyl | cooking | | |
| | temperature | chloride addition | temperature | Yield | Purity |
| S. No | ° C. | temperature ° C. | ° C. | % | % |
| 1 | 0 to 5 | 10 to 15 | 0 to −5 | 77.5 | 94 |
| 2 | 0 to 5 | 10 to 15 | 10 to 15 | 98 | 96 |
| 3 | 0 to 5 | 10 to 15 | 30 to 35 | 77 | 94.4 |
| 4 | 0 to 5 | 10 to 15 | 20 to 25 | 84 | 94 |

### Preparation of Compound 1 by Varying the Solvent and the Scavenger

The benzoxazinone compound 1 of the present disclosure was prepared as provided in Example-1 and by varying the solvent and the scavenger as shown in Table-4 below. The combination of appropriate solvent and the scavenger provided a higher purity and increased yield of the compound 1 of the present disclosure. It can be seen that acetonitrile as the solvent resulted in a higher yield and higher purity whereas dichloroethane resulted in decreased yield, but higher purity. Similarly use of tetrahydrofuran, ethyl acetate caused decreased yield yet an appreciable purity. Whereas dimethylformamide with 3-picoline or pyridine as scavenger resulted in higher yield and purity but with triethyl amine caused lower yield. Hence it could be deduced that the choice of solvent with the scavenger is significant in the determining the desired yield and purity of the compound of Formula I prepared by the process of the present disclosure.

TABLE 4

| S. No | Solvent | Scavenger | Yield % | Purity % |
|---|---|---|---|---|
| 1. | Acetonitrile | Pyridine | 95 | 97 |
| 2. | Acetonitrile | 3-Picoline | 98 | 97 |
| 3. | Acetonitrile | Triethyl amine | 88 | 95 |
| 4. | 1,2-Dichloroethane | 3-Picoline | 57 | 96 |
| 5. | 1,2-Dichloroethane | Pyridine | 51 | 96 |
| 6. | Tetrahydrofuran | 3-Picoline | 52 | 95 |
| 7. | Ethyl acetate | 3-Picoline | 76 | 96 |
| 8. | Acetone | Pyridine | 90 | 88 |
| 9. | Dimethylformamide | 3-Picoline | 88 | 89 |
| 10. | Dimethylformamide | Pyridine | 90 | 86 |
| 11. | Dimethylformamide | Triethyl amine | 50 | 81 |

### Comparative Impurity Studies on the Addition of Scavenger

Compound 1 of the present disclosure is prepared by the process defined herein wherein the scavenger and the reagent is added in a single lot. For the purpose of comparison, the process disclosed in WO03015519 was carried out which included two lot addition of scavenger and reagent. Nevertheless the process of the present disclosure resulted in higher purity and yield, and further the impurities formed in process of the present disclosure were also analysed. Similarly the compound 1 was prepared by the process as defined in WO03015519, wherein the scavenger and reagent are added in two lot in a different reaction sequence and the impurities/product obtained were analysed. The compound 1 was prepared by the processes, involved acetonitrile as solvent, methane sulfonyl chloride as reagent and pyridine as scavenger. The result obtained regarding the impurity profile is also illustrated below in Table-5.

TABLE 5

| | |
|---|---|
| 2 lot addition of | 1 lot addition of scavenger |
| scavenger and reagent | and reagent (pyridine and |
| (pyridine and methane | methane sulphonyl |
| sulphonyl chloride) | chloride) (present |
| (WO03015519) | invention) |

| S. No | HPLC -RT | % Area | HPLC -RT | % Area |
|---|---|---|---|---|
| 1 | 4.805 | 0.28 | 5.288 | 0.1 |
| 2 | 6.822 | 0.11 | 8.224 | 0.47 |
| 3 | 8.339 | 0.74 | 8.395 | 0.84 |
| 4 | 8.688 | 95.2 | 8.748 | 97.2 |
| 5 | 9.011 | 0.36 | 9.072 | 0.31 |
| 6 | 9.092 | 3.000 | 9.152 | 0.87 |
| 7 | 9.504 | 0.11 | — | — |
| 8 | 9.657 | 0.11 | — | — |

From Table-5 it can be understood that the two lot addition of the scavenger as described in the process of WO03015519 resulted in the formation of 7 impurities, the outcome was the reduction in the yield of compound 1 to 75% (above the % of area 0.1 in HPLC). In contrast the process of the present disclosure, wherein the scavenger and the reagent was added in single lot resulted only in 5 impurities, and compound 1 with ˜95% of yield and 97% purity. Based on the peak area in HPLC, the % of the impurities, the type and number of the impurities formed were determined and compared. The number and the type of the impurities were found to be different in both the processes as evident from the Table-5. Further it is to be noted that the purity and yield of the compound 1 formed in the present disclosure is higher than the compound 1 formed using the process of WO03015519. Hence it could be deduced that the method and sequence of addition of scavenger is essential and significant in obtaining highly pure compounds of Formula I.

Similarly, Table-6 below illustrates the preparation of the compound 1 using the process of the present disclosure, and as per the process defined in WO03015519 with 3-picoline as the scavenger. The results obtained are similar to the results obtained with pyridine as scavenger.

TABLE 6

| | | | 1 lot addition of scavenger | |
|---|---|---|---|---|
| | 2 lot addition of scavenger | | and reagent (3-picoline and | |
| | and reagent (3-picoline | | methane sulphonyl | |
| | and methane sulphonyl | | chloride) (present | |
| | chloride) (WO03015519) | | invention) | |
| S. No | HPLC -RT | % Area | HPLC -RT | % Area |
| 1 | 5.138 | 0.13 | 5.163 | 0.1 |
| 2 | 8.332 | 0.71 | 8.378 | 0.64 |
| 3 | 8.434 | 0.16 | — | — |
| 4 | 8.687 | 95.2 | 8.735 | 97.1 |
| 5 | 9.018 | 0.26 | 9.065 | 0.25 |
| 6 | 9.104 | 3.290 | 9.145 | 1.5 |
| 7 | 9.676 | 0.1 | — | — |

The process of present disclosure resulted in a higher purity of the compound of Formula I and the yield and purity was highly dependent on the various process parameters as illustrated above. Thus it is necessary to maintain the process parameters and the process sequence along with sequential addition of the scavengers, reagents and solvent as disclosed herein to obtain pure compounds of Formula I. Any slight deviation eventually affects the purity of the compounds obtained and their yield. Also the present disclosure illustrated the necessity of maintaining the molar ratio of the reacting compounds, reagents, and scavengers intact for maximizing the advantages over the existing prior arts.

Further the compounds of Formula I are useful intermediates in preparing agrochemicals. Therefore, the purity of 97% and above of the intermediates is an influential factor in obtaining high pure agrochemicals. The higher purity of the intermediates ensures higher purity of the agrochemicals and thereby decreases the cost of production of the high pure agrochemicals. The minimum the impurities formed, minimum will be the time and manpower involved to obtain high pure agrochemicals. Also ensuring purity throughout the synthetic process will bring down the overall expenditure and also paves way for large scale production without any hindrance. The necessity to separate the impurities shall be avoided and a continuous production of the desired products becomes evident.

Moreover, the current need in producing agrochemicals of high purity, in turn implies environmental responsibility. The impure products poses problems related to biodegradability, environmental accumulation and ultimately turning toxic to humans and other organisms. The environmental and toxicity issues due to impurities are threat and would continue to exist for long time even after the use of the agrochemicals, hence agrochemicals with reduced impurities is an ultimatum to prevent such occurrences. Therefore the process of the present disclosure provides a high pure compound of Formula I which are intermediates in preparing agrochemicals, and agrochemicals prepared from the compounds of Formula I are also of high purity.

### Advantages of the Present Disclosure

The present disclosure provides a process for preparing the compounds of Formula I. The process of the present disclosure is a simple and cost effective process. The process of the present disclosure involves simple one step addition of the reacting materials, the reagent, and the scavenger. The process of the present disclosure is one-pot synthetic process which is industrially scalable. The process involves ambient temperature and pressure conditions and does not require very low temperature, thereby the process provides feasible operating conditions. The reagents and scavenger are added in single lot which leads to easy handling of the reaction process. The process of the present disclosure is energy saving and cost saving as it involves single lot addition of the reagents, ambient reaction temperature and requires lesser time than the conventional processes. The process of the present disclosure also results in a higher yield in the range of 80 to 98% with purity in the range 90 to 99%, hence is an efficient preparation process.

I/We claim:

1. A process for preparing a compound of Formula I, 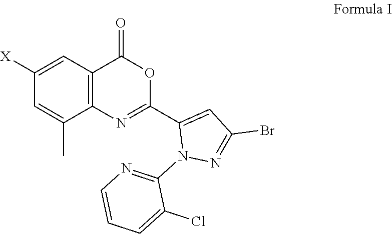
the process comprising:
a. mixing a compound of Formula II with a compound of Formula III in a mole ratio range of 1:1 to 1.3:1 to obtain a first mixture;
b. adding a solvent to the first mixture to obtain a second mixture followed by cooling the second mixture;
c. contacting a scavenger with the second mixture to obtain a reaction mass; and
d. heating the reaction mass followed by addition of a reagent to obtain the compound of Formula I, 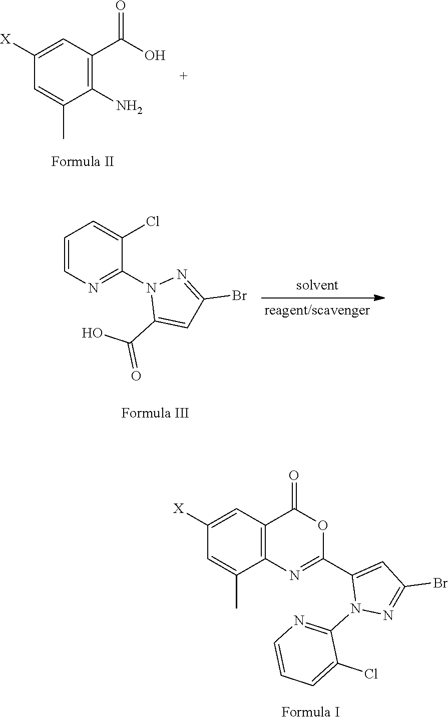
wherein X is selected from Cl, Br, I, or CN; and
the scavenger in step c and the reagent in step d are added in a single lot.

2. The process as claimed in claim 1, wherein the solvent is selected from acetonitrile, ethylene dichloride, tetrahydrofuran, ethylacetate, acetone, dimethyl formamide, or combinations thereof.

3. The process as claimed in claim 1, wherein the scavenger is selected from triethyl amine, 3-methyl pyridine, 2-methyl pyridine, pyridine, 4-dimethylaminopyridine, or combinations thereof.

4. The process as claimed in claim 1, wherein the reagent is selected from methane sulfonyl chloride, tosyl chloride, or oxalyl chloride.

5. The process as claimed in claim 1, wherein cooling the second mixture is carried out at a temperature in a range of 0 to 10° C.

6. The process as claimed in claim 1, wherein heating the reaction mass is carried out at a temperature in a range of 10 to 20° C.

7. The process as claimed in claim 1, wherein the reagent is slowly added to the reaction mass for a time period in a range of 1 to 2.5 hours at a temperature in a range of 10 to 20° C.

8. The process as claimed in claim 1, wherein the compound of Formula III, the scavenger and the reagent are in a mole ratio range of 1:4:2 to 1:6:2.5.

9. The process as claimed in claim 1, wherein the compound of Formula I is obtained with yield in a range of 80 to 98%; and purity in a range of 90 to 99%.
